(19) United States
(12) Patent Application Publication
(10) Pub. No.: US 2023/0248654 A1
(43) Pub. Date: Aug. 10, 2023

(54) SWELLABLE ORAL PHARMACEUTICAL COMPOSITIONS

(71) Applicant: Adare Pharmaceuticals, Inc., Lawrenceville, NJ (US)

(72) Inventors: Nathan DORMER, Lawrenceville, NJ (US); Michelle K. SCHILLING, Lawrenceville, NJ (US); Jeffrey OVERFIELD, Lawrenceville, NJ (US)

(21) Appl. No.: 18/015,192

(22) PCT Filed: Jul. 12, 2021
(86) PCT No.: PCT/US2021/041277

§ 371 Date: Jan. 9, 2023

Related U.S. Application Data
(60) Provisional application No. 63/050,363, filed on Jul. 10, 2020.

(51) Int. Cl. A61K 9/20

(52) U.S. Cl. CPC A61K 9/2054; A61K 9/2018; A61K 9/2077

(57) ABSTRACT

Swellable oral compositions are described which, upon addition of water, form semi-solid products prior to administration. The compositions are particularly suitable for administration to subjects who may have difficulty in, or a dislike for, swallowing solid oral compositions, such as tablets and capsules.

### FIELD

This disclosure relates to swellable oral compositions which, upon addition of water, form semi-solid products prior to administration. The compositions are particularly suitable for administration to subjects who may have difficulty in, or a dislike for, swallowing solid oral compositions, such as tablets and capsules.

### BACKGROUND

Tablets and capsules are the most widely used dosage forms for oral drug administration. However, these dosage forms have several disadvantages. For example, it is estimated that 50% of the population have problems swallowing tablets. Many aged people find it especially difficult to swallow tablets or capsules, and the medication of children is compromised when subjects are unable or unwilling to swallow tablets or capsules. This leads to poor compliance with treatment regimens, which can negatively impact the efficacy of the drug administered. Furthermore, many therapeutic agents have a bitter taste, precluding them from being sprinkled onto food, such as applesauce, which is a commonly used method of administering medications to children.

The present disclosure solves these and other problems associated with solid oral dosage forms by providing a solid pharmaceutical product which is readily and rapidly converted into a semi-solid form just before administration following the addition of a small amount water. The semi-solid product is more palatable to individuals unable, or unwilling, to take solid oral dosage forms, thus improving compliance and ensuring the appropriate drug regimen is administered to treat or cure ailments.

U.S. Pat. Nos. 8,383,154 and 8,383,155 describe the application of Parvulet® technology to produce swellable oral compositions which, upon addition of water, form semi-solid products. However, this technology has some limitations. The technology is particularly suited to powder compositions. Also, for some drugs, the Parvulet® technology may need to be modified to satisfactorily control drug dissolution and release rates.

### SUMMARY

The main objective of this disclosure is to provide a new drug dosage form with enhanced patient acceptance. The new solid pharmaceutical products of this disclosure facilitate a convenient method of orally administering drugs that gives patients expanded optionality for adhering to treatment regimens in situations where compliance with administration of traditional oral dosage forms, such as tablets or capsules, is compromised due to a bitter taste or swallowing difficulty. The new pharmaceutical products described herein also include controlled release products that can effectively control a drug's dissolution rate and release properties.

The present disclosure therefore provides a solid pharmaceutical product for oral administration comprising a drug-containing component and a swellable component. The product is fully converted to a semi-solid form, such as a semi-solid gel, following the addition of a small amount of water. The semi-solid form may be produced without applying shear forces or other mixing forces, and is easier to swallow than conventional dosage forms, such as tablets or capsules.

The present disclosure also provides a combination of drug-containing and swellable components which allows for the preparation of robust tablets with properties which facilitate their complete conversion to a semi-solid form (e.g. gel) within minutes by the addition of a small amount of water. The conversion may be achieved without stirring, shaking, heating or any other method of mixing or applying shear forces. One particular advantage of the tablets described herein is that their preparation may be readily scaled up for commercial manufacture.

### BRIEF DESCRIPTION OF THE DRAWINGS

FIG. 1 shows photographs of the product of Example 1 before and after the addition of water.

FIG. 2 shows the dissolution of the product of Example 1 compared to the dissolution of the drug microsphere component alone.

FIG. 3 shows a photograph of the product of Example 2 after the addition of water.

FIG. 4 shows the dissolution of the product of Example 2 compared to the dissolution of the drug microsphere component alone.

FIG. 5 shows the release profile of the product of Example 3 in pH 5.8 phosphate buffer.

FIG. 6 shows the release profile of the product of Example 4 in pH 5.8 phosphate buffer.

### DETAILED DESCRIPTION

The present disclosure provides a solid pharmaceutical product comprising a drug-containing component and a swellable component. The disclosure also provides methods for making the product, conversion of the product to a semi-solid form (e.g. gel) and methods for administering the semi-solid form to patients. The product may conveniently be presented as a powder or granules, for example, packaged in a sachet for use, or may be a robust tablet with properties that allow for its rapid complete conversion to a semi-solid product following the addition of a small amount of water.

### Definitions

The term “drug”, “active” or “active pharmaceutical agent” as used herein includes a pharmaceutically acceptable and therapeutically effective agent and any pharmaceutically acceptable salts, racemate, enantiomer thereof. It may be chosen from several pharmaceutical categories: such as antimicrobials, analgesics, anti-diabetics, anti-inflammatory, neurolepic agents, antipsycotics, carbamic anhydrase inhibitors, antiallergic, antiasthmatic, antihistaminic, proton pump inhibitors, steroids, corticosteroids, anticonvulsants, antiepileptics, broncodilators, hypnotics, expectorants, mucolytics, anticancer, anti-hyperlipidemics cardiovascular, gingipain inhibitors, antibiotics, antivirals, vitamins, minerals, peptides, enzymes, proteins, oligonucleotides, biologics, probiotics etc. The drug may be lipophilic or hydrophilic. It may be a drug of Class II, including a drug susceptible for abuse (i.e. known to have properties that can lead to addiction).

The term “controlled release” as used herein include the terms extended release, modified release, delayed release, sustained release, or immediate release.

As herein used, the singular forms “a”, “an”, and “the” include plural references unless the content clearly dictates otherwise. Thus, for example, “a water-soluble polymer” includes a mixture of one or more water-soluble polymers.

The term “about”, as used herein to refer to a numerical quantity, includes “exactly”. For example, “about 60 seconds” includes 60 seconds, exactly, as well as values close to 60 seconds (e.g., 50 seconds, 55 seconds, 59 seconds, 61 seconds, 65 seconds, 70 seconds, etc.). In some instances, the term “about” in the context of the disclosure refers to an approximate amount distinct from adjacent values. For example, in a disclosure of “about 65, about 70, about 75,” “about 70” refers to an amount as low as a value greater than 65.5 or less than 74.5. When the term “about” is used in reference to a range of values, the term “about” refers to both the minimum and maximum value of the range (e.g., “about 1-50 μm” means “about 1 μm to about 50 μm”).

Unless indicated otherwise, all percentages and ratios are calculated by weight based on the total weight of component, or of the composition, or of the dosage form.

Throughout the present document, all expressions of percentage, ratio, and the like, are in weight units unless otherwise indicated.

The term “drug-containing component” includes a plurality of drug microparticles or drug mini-tablets.

“Drug microparticles” are drug-containing solids having a particle diameter size in the micrometer range, and may include, for example, microspheres, microcapsules, beads, granules, pellets or micro-tablets. A plurality of drug microparticles may conveniently be present in powder form.

“Mini-tablets”, “Minitabs” or “MMTS™” (Multi MiniTablet System) are tablets with a diameter of about 1 mm to about 2 mm. The mini-tablets may comprise one or more drugs and excipients.

“Effective amount” or “therapeutically effective amount”, as used herein, means the amount of the drug to be dosed once or multiple times daily in a patient with the disorder to cause the desired therapeutic effect.

### Objects of the Present Disclosure

A first object of the present disclosure is a solid pharmaceutical product comprising a drug-containing component and a swellable component, wherein the product is fully converted to a semi-solid form within about 2 minutes following the addition of water, without applying shear forces or other mixing forces.

A second object of the present disclosure is a tablet comprising a drug-containing component and a swellable component, wherein the tablet is fully converted to a semi-solid form within about 2 minutes following the addition of water, without applying shear forces or other mixing forces.

### Drug-Containing Component

The drug-containing component of the present disclosure may include a plurality of drug microparticles which can control the release of the drug from the pharmaceutical composition. Alternatively, the drug-containing component of the present disclosure may include a plurality of drug mini-tablets, which may be coated or uncoated.

In one embodiment of the disclosure, the microparticles are microspheres. In a particular embodiment, the microspheres comprise of one or more waxes, lipids, celluloses, and other excipients such as controlled release agents, with drug content ranging from about 1% w/w to about 90% w/w.

The microspheres may, in one embodiment, provide for an extended release of the drug. Such “extended release microspheres” can also be combined with a free or “immediate” fraction of drug, such that the patient benefits from a rapid initial dose in combination with an extended (e.g. “all day”) dose. An immediate fraction of drug is a drug component that releases the drug immediately and more than about 85% of drug release occurs in 30 minutes after administration.

In one embodiment of the disclosure, the microparticles are microcapsules comprising a core/shell structure, wherein the core portion comprises a drug and an excipient, and wherein the shell portion encapsulating the core comprises a hydrophobic matrix and a pH-responsive material.

Controlled release microparticles can be manufactured according to different methods including, but not limited to, melt spray congeal, prilling, spray chilling, spray drying, spinning disc, hot melt extrusion, melt granulation, fluid bed coating, Wurster coating, pan coating, extrusion spheronization, emulsion, or coacervation.

In one aspect, microparticles are manufactured by a melt spray congeal process, which utilizes an accelerating co-flowing gas stream, combined with piezoelectric vibration.

The microparticles of the present disclosure may also be prepared using Optimμm® technology.

Examples of the preparation of microcapsules are described in U.S. Pat. No. 10,426,734, US 20160354317, and US 2019035655.

In one embodiment, the microparticles, including microcapsules, of this disclosure have an average particle size (diameter) of from about 90 μm to about 1000 μm (e.g. measured with Malvern particle size analyzer), have a generally spherical shape and a narrow particle size distribution.

In one embodiment, the drug microparticles are micro- and nano-spheres having an average particle size (diameter) of about 50 μm to about 100 μm. Ninety percent of these particles have a diameter that is within 2% of an average diameter of the particles. The preparation of these particles is carried out with the Optimμm® technology as described in U.S. Pat. Nos. 6,669,961, 7,368,130, 8,409,621 and 7,309,500.

In one embodiment of the present disclosure, the microparticles have an average particle size (diameter) of from about 50 μm to about 300 μm.

In a particular embodiment, the microparticles have an average particle size (diameter) of from about 50 μm to about 300 μm and comprise a hydrophobic matrix material. The microparticles may also contain other excipients, such as a stabilizer and/or a release modifier. Such microparticles and their preparation are described in U.S. Pat. No. 10,398,649. These particles are particularly suitable for use with hydrophilic drugs.

In one particular embodiment, the microparticles have an average particle size of 200 μm or less (e.g. about 150 μm to about 200 μm). Such particles may conveniently have polymeric coating. For example, the microparticles may have an average particle size of about 150 μm to about 200 μm and a polymeric coating of about 10% to about 20% w/w.

In one particular embodiment of this disclosure, the microparticles are microcapsules having an average particle size of 200 μm or less. Such particles may conveniently have a coating, e.g. an ethylcellulose coating. Such microcapsules may be prepared by standard methods, or by coacervation. In one example, microcapsules having an average particle size of 200 μm or less and having an ethylcellulose coating are prepared by coacervation with ethylcellulose in cyclohexane.

In one embodiment of the disclosure, the microparticles are mini-tablets. The mini-tablets have an average diameter of about 2 mm or less, e.g. from about 1 mm to about 2 mm, and particularly about 2 mm. The mini-tablets may be produced on conventional tablet presses equipped with multiple tooling. Mini-tablets production is similar to the production of standard tablets, but requires excellent powder flow due to the small dies, exact control of process parameters and special caution during tablet press assembly in order to avoid tool damage. Mini-tablets may be coated or uncoated. Mini-tablets (or Minitabs®) may be prepared using the same excipients as those described hereinafter for regular sized tablets.

In some embodiments, the drug is present an amount ranging from about 30% to about 60% by weight of the total mass of the microparticle, e.g. about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, 50%, about 51%, about 52%, about 53%, about 54%, about 55%, about 56%, about 57%, about 58%, about 59%, or about 60%, including each value and subrange between these values.

In one specific embodiment, the drug is lipophilic, and the drug loading is up to 50%.

### Swellable Component

The swellable component of the present disclosure provides fluidity and palatability to the pharmaceutical composition at ambient temperature when mixed to water. The swellable component may conveniently be prepared as described in U.S. Pat. Nos. 8,383,154 and 8,383,155.

In one aspect of the present disclosure, the swellable component comprises one or more swellable hydrophilic polymers.

In another aspect of the present disclosure, the swellable component comprises one or more swellable hydrophilic polymers and one or more hydrophilic agents. The hydrophilic agent(s) may be added to the swellable component to improve the swelling properties of the swellable hydrophilic polymer(s) and ultimately the solid pharmaceutical product.

In one embodiment of the above aspects, the one or more swellable hydrophilic polymers constitute about 20% to about 80% by weight of a total swellable component.

The hydrophilic polymers herein are capable of forming a highly viscous material or a gel by addition of water. They are preferably hydrocolloids such as gellan gum, agar, alginate, carrageenan, locust beam gum, cellulose derivatives, starch derivatives. In one embodiment, the swellable hydrophilic polymer is gellan gum. In a specific embodiment the swellable hydrophilic polymer is high acyl gellan gum or it is a gellan gum acylated within a degree of up to 4 per every two repeats of the glucose-rhamnose-glucose-glucuronic acid unit of the polymer.

The hydrophilic agent(s) may conveniently be selected from the group consisting of electrolytes, organic acids and osmotic agents, and mixtures thereof.

One class of suitable osmotic agents include osmo-polymers such as hydrophilic vinyl and acryl polymers, polysaccharides, polyethylene oxide (PEO), polyethylene glycol (PEG), polypropylene glycol (PPG), poly(2-hydroxyethylmethacrylate), poly(acrylic)acid, poly(methacrylic)acid, polyvinylpyrrolidone (PVP), polyvinyl alcohol (PVA), PVA/PVP copolymers, hydroxy ethyl cellulose (HEC), hydroxy propyl cellulose (HPC), hydroxypropyl methyl cellulose (HPMC), carboxymethyl cellulose (CMC), carboxyethyl cellulose (CEC), sodium alginate, polycarbophil, gelatin and sodium starch glycolate. Another class of suitable osmotic agent include osmotically effective solutes (osmogens) such as water-soluble organic acids, salts and sugars; magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, inulin, instant sugar, citric acid, succinic acid and tartaric acid.

In one aspect of the present disclosure, the swellable component comprises a gellan gum (e.g. high acyl gellan gum) and one or more hydrophilic agents.

In one aspect of the present disclosure, the swellable component comprises one or more swellable hydrophilic polymers and a hydrophilic agent which is an osmogen.

In one aspect of the present disclosure, the swellable component comprises a gellan gum (e.g. high acyl gellan gum) and a hydrophilic agent which is an osmogen.

In one embodiment of the above aspects, the osmogen is a natural sugar or sugar substitute.

In a particular embodiment of the above aspects, the osmogen is selected from mannitol, and lactose.

In a further particular embodiment, the swellable component comprises gellan gum (e.g. high acyl gellan gum), a hydrophilic agent selected from lactose and mannitol and a compression aid. In a preferred embodiment the swellable component comprises gellan gum (e.g. high acyl gellan gum), lactose and silicified microcrystalline cellulose.

Examples of hydrophilic electrolytes include ionizable substances such as monovalent, divalent or multivalent ionizable salts. The salts may, for example, be selected from inorganic salts, including various alkali metal and/or alkaline earth metal sulfates, chlorides, borates, bromides, etc., and ionizable alkaline earth organic salts such as citrates, acetates, lactates etc., calcium sulfate or sodium chloride. Examples of hydrophilic organic acids include benzoic acid, succinic acid, citric acid and adipic acid.

In a particular embodiment, the swellable component comprises gellan gum (e.g. high acyl gellan gum), lactose, calcium citrate and silicified microcrystalline cellulose. In another particular embodiment, the swellable component comprises gellan gum (e.g. high acyl gellan gum), mannitol, calcium citrate and silicified microcrystalline cellulose.

Conveniently, the swelling component may be in the form of a powder or granulate prior to mixing with the drug-containing component. However, when the drug-containing component comprises a plurality of mini-tablets, the swelling component may similarly be presented as a plurality of mini-tablets prior to dry blending with the drug-containing component mini-tablets. The swelling component mini-tablets will conveniently have approximately the same dimensions as the drug containing component mini-tablets and are prepared in a similar manner. Thus, the swelling component mini-tablets have an average diameter of about 2 mm or less, e.g. from about 1 mm to about 2 mm, and particularly about 2 mm. These mini-tablets may be produced on conventional tablet presses equipped with multiple tooling and may be coated or uncoated. Swelling component mini-tablets (or Minitabs) may be prepared using the same excipients as those described hereinafter for regular sized tablets.

In a particular embodiment, the swellable component mini-tablets comprise gellan gum (e.g. high acyl gellan gum), a hydrophilic agent selected from lactose and mannitol and a compression aid such as silicified microcrystalline cellulose. In a preferred embodiment, the swellable component mini-tablets comprise gellan gum (e.g. high acyl gellan gum), lactose and silicified microcrystalline cellulose.

### Solid Pharmaceutical Product

A solid pharmaceutical product of the present disclosure comprises a drug-containing component and a swellable component, wherein the product is fully converted to a semi-solid form within about 2 minutes following the addition of water, without applying shear forces or other mixing forces.

The drug-containing component may include a plurality of microparticles or mini-tablets as described hereinabove. Drug microparticles may conveniently be present at a concentration of about 1% to about 80% w/w in the final product. Similarly, drug mini-tablets may conveniently be present at a concentration of about 1% to bout a80% w/w in the final product.

The type of microparticles and their ingredients dictates the drug release kinetics and other patient-centric benefits (e.g. taste masking). The microparticles can be designed to release drug at a slow or at a fast rate, such that a patient can benefit from therapeutic effect without poor taste or a large pill to ingest.

The solid pharmaceutical product swells and forms a semi-solid mass/gel within about 2 minutes following the addition of a pre-determined amount of water, without applying any shear forces or other mixing forces to encourage the formation of a homogeneous semi-solid mass/gel.

In one embodiment of the present disclosure, the solid pharmaceutical product is prepared as a loose powder by dry blend mixing of the drug-containing component and the swellable component. The powder may conveniently be packaged in the form of a sachet prior to use. For oral administration, the sachet will be opened and the powder contents poured onto a dispensing agent, such as a spoon. Following the addition to the powder of a small, pre-determined, amount of water sufficient to form a semi-solid mass/gel without any powder product remaining, the patient may swallow the mass/gel. The mass/gel should have a smooth consistency which allows the subject to swallow the product without any discomfort. In a particular embodiment, the semi-solid mass/gel is formed within about 60 seconds, particularly within about 45 seconds, and preferably within about 30 seconds, following the addition of a small pre-determined, amount of water to the solid pharmaceutical product in powder form.

In one embodiment of the present disclosure, the solid pharmaceutical product is prepared as a tablet by first forming a dry mixture of the drug-containing components and the swellable components and then compressing the resulting mixture to form the tablet using conventional tableting methods. In a particular embodiment, the so-formed tablet exhibits a tablet friability of 1% or less, e.g. about 0.5% or less. In a further embodiment, the tablet is fully converted to a semi-solid form (e.g. gel) within about 2 minutes, preferably within about 90 seconds, following the addition of a small, pre-determined amount of water. In a particular embodiment, the tablet is fully converted to a semi-solid form (e.g. gel) within about 45 seconds following the addition of a small, pre-determined amount of water.

The applicant has found that the inclusion of a compression aid in the solid pharmaceutical product prior to tableting is advantageous for producing tablets having the following combination of features: (1) tablet friability ≤1% (e.g. ≤0.5%) (2) a tablet hardness value (N) such that the tablet is sufficiently robust for large-scale manufacture and packaging and (3) the tablet is fully converted to a semi-solid form (e.g. gel) within about 2 minutes following the addition of a small, pre-determined amount of water.

Furthermore, surprisingly, the inclusion of a compression aid in combination with particular osmogens in the swelling component results in improved tablet features (1), (2) and (3).

Examples of suitable compression aids include microcrystalline celluloses, such as silicified microcrystalline cellulose (e.g. SMCC90 Prosolv).

Osmogens of particular interest for use in the tablet products herein include sugars such as mannitol (e.g. Mannogem 2028 or Partek M200) and lactose (e.g. Capsulac 60 or Lactose mono).

In a specific embodiment, the present disclosure provides a tablet comprising a drug-containing component and a swellable component, wherein the tablet comprises gellan gum, a compression aid (e.g. silicified microcrystalline cellulose) and an osmogen selected from mannitol (e.g Mannogem 2028 or Partek M200) and lactose (e.g. Capsulac 60 or Lactose mono). In an even more specific embodiment, the present disclosure provides a tablet comprising a drug-containing component and a swellable component, wherein the tablet comprises gellan gum, lactose (e.g. Capsulac 60 or Lactose mono) as the osmogen, and silicified microcrystalline cellulose as the compression aid. In a preferred embodiment, said tablet is fully converted to a semi-solid form within about 2 minutes, e.g. within 45 seconds, following the addition of water, without applying shear forces or other mixing forces.

In a specific embodiment, drugs which may be included in a solid pharmaceutical product of the present disclosure include ibuprofen, cetirizine and acetaminophen (APAP).

In a specific embodiment, ibuprofen microparticles can be combined with the swellable component at a concentration of 20-65% or 50-65% w/w in the final product; in a preferred embodiment it amounts to 62% w/w.

In a specific embodiment, cetirizine microparticles can be combined with the swellable component at a concentration of 10-50% w/w in the final product; in a preferred embodiment it amounts to 30% w/w.

In a specific embodiment, acetaminophen (APAP) microparticles can be combined/blended with the swellable component at a concentration of 10-50% w/w in the final product. In a preferred embodiment it amounts to 24% w/w or to 30% w/w or to 36% w/w.

In one specific embodiment, the solid pharmaceutical product comprises cetirizine microparticles in amount of about 30% w/w based on the total weight of said composition and exhibiting a size distribution with a D[4,3]˜250 μm, and further comprising a swellable component which includes gellan gum (high acyl) in amount of about 28% w/w in the swellable component, wherein the swellable component further comprises calcium citrate in combination with mannitol.

In one specific embodiment, the solid pharmaceutical product comprises acetaminophen microparticles in an amount of 24%, or 30%, or 36% w/w based on the total weight of said composition, and further comprising a swellable component which includes gellan gum (high acyl) in amount of about 15-30%, preferably 20% or 25%, w/w based on the total weight of said product.

In one embodiment of the disclosure, the solid pharmaceutical product comprises acetaminophen microparticles in the amount of about 24% w/w based on the total weight of said product, and further comprising a swellable component which includes gellan gum (high acyl) in an amount of about 20% w/w based on the total weight of said product, wherein the swellable component further comprises lactose.

In one specific embodiment of the disclosure, the solid pharmaceutical product comprises ibuprofen microparticles in an amount of about 62% w/w based on the total weight of said product and having a size distribution with a D[4,3]˜250 μm, and further comprising the swellable component which includes gellan gum (high acyl) in an amount of about 52% w/w of the swellable component, and wherein the swellable component further comprises calcium citrate.

### Embodiments

EMBODIMENT 1: A controlled release pharmaceutical composition comprising a drug microparticles component and a swellable component.

EMBODIMENT 2: The composition of EMBODIMENT 1, wherein the drug microparticles are incorporated in the swellable component which comprises at least one swellable hydrophilic polymer.

EMBODIMENT 3: The composition of EMBODIMENT 1 or EMBODIMENT 2, wherein the swellable component further comprises a hydrophilic agent.

EMBODIMENT 4: The composition of any of the preceding EMBODIMENTS, wherein the swellable hydrophilic polymer is gellan gum-high acyl.

EMBODIMENT 5: The composition of any of the preceding EMBODIMENTS, wherein the drug microparticles are present at a concentration of 1% to 80% w/w of the total weight of the composition.

EMBODIMENT 6: The composition of any of the preceding EMBODIMENTS, wherein the drug microparticles are present in amount of about 1-80% w/w of the total weight of the composition, and the hydrophilic polymer is present in in amount of about 20-80% w/w of the swellable component.

EMBODIMENT 7: The composition of any of the preceding EMBODIMENTS, wherein the drug microparticles are microspheres having an average diameter of 50 to 100 μm as measured with Malvern and ninety percent of these particles have a diameter that is within 2% of an average diameter of the particles.

EMBODIMENT 8: The composition of any one of EMBODIMENTS 1-6, wherein the drug microparticles have a particle having a particle diameter from about 50 to about 300 μm, comprising a hydrophobic matrix material, a stabilizer and a release modifier.

EMBODIMENT 9: The composition of any one of EMBODIMENTS 1-6, wherein the drug microparticles are microcapsules comprising a core-shell-structure, wherein the core portion comprises the drug and an excipient, and wherein the shell portion encapsulating the core comprises a hydrophobic matrix.

EMBODIMENT 10: The composition of any one of the preceding EMBODIMENTS, wherein the composition is in form of tablet, powder, capsule, sachet.

EMBODIMENT 11: The composition of any one of the preceding EMBODIMENTS, wherein the drug is ibuprofen, wherein the drug microparticles are present in amount of 50-65% w/w of the composition and have a size distribution with a D[4,3]˜250 μm, wherein the gellan gum (HA) is in amount of about 20-80% w/w of the swellable component and wherein the swellable component comprises calcium citrate.

EMBODIMENT 12: The composition of any one of EMBODIMENTS 1-10, wherein the drug is cetirizine, wherein the drug microparticles are present in amount of 10-50% w/w of the composition and have a size distribution with a D[4,3]˜250 μm, wherein the gellan gum (HA) is in amount of about 20-80% w/w of the swellable component and wherein the swellable component comprises calcium citrate and mannitol.

EMBODIMENT 13: The composition of any one of EMBODIMENTS 1-6 or 10, wherein the drug microparticles have particle size of below 200 μm.

EMBODIMENT 14: The composition of one of EMBODIMENTS 1-6 or 10, wherein the drug microparticles have particle size of below 200 μm and are coacervated microcapsules with ethylcellulose.

EMBODIMENT 15: The composition of EMBODIMENT 14 wherein the drug is acetaminophen (APAP), wherein the drug microparticles are present in amount of about 10-50% of the composition, wherein the gellan gum (HA) is in amount of about 15-30% w/w of the composition and wherein the swellable component comprises lactose.

EMBODIMENT 16: Method of administering to a patient the controlled release pharmaceutical composition of any one of the preceding EMBODIMENTS after addition of an aqueous medium without applying shear forces.

The following examples illustrate solid pharmaceutical products of the present disclosure.

### EXAMPLES

### Example 1. Preparation of Extended-Release Ibuprofen Sachet

To make the drug component (microspheres), a molten solution consisting of 64% carnauba wax, 25% ibuprofen, 10% stearic acid, and 1% ethylcellulose was mixed under stirring at 100° C. The molten solution was then processed via a melt spray congeal process, which utilizes an accelerating co-flowing gas stream, combined with piezoelectric vibration. The resulting cooled powder exhibited a size distribution with a D[4,3]˜250 μm.

To make the swellable component, 38.9% SMCC Prosolv®, 52.1% gellan gum (HA), 7.9% calcium citrate, and 1.1% D&C Red #7 were high-shear granulated.

To make the pharmaceutical composition, the microsphere component was dry blended with the swellable component; such that the microspheres constituted 62% of the dosage form weight, corresponding to a 200 mg ibuprofen dose.

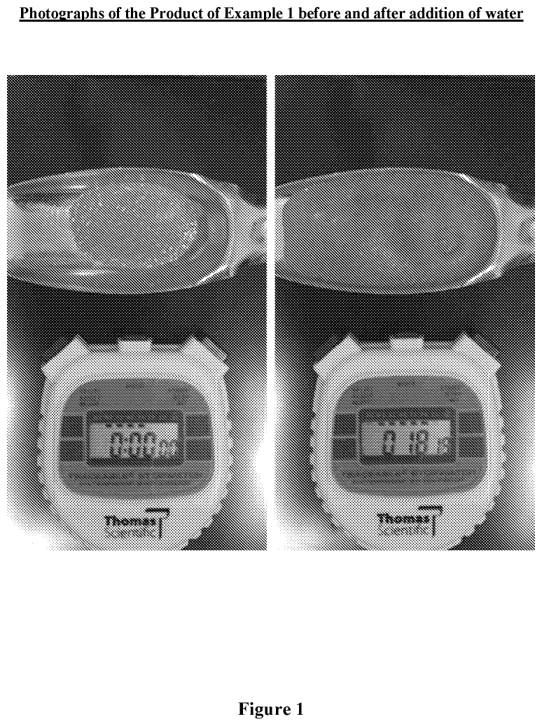

For swell testing of the composition, 10 mL of tap water was added to the dry pharmaceutical powder laid down on a spoon to create the palatable matrix with an apple sauce-like consistency over an 18 second duration (n=10). Photographs of the product before and after the addition of water are displayed in FIG. 1.

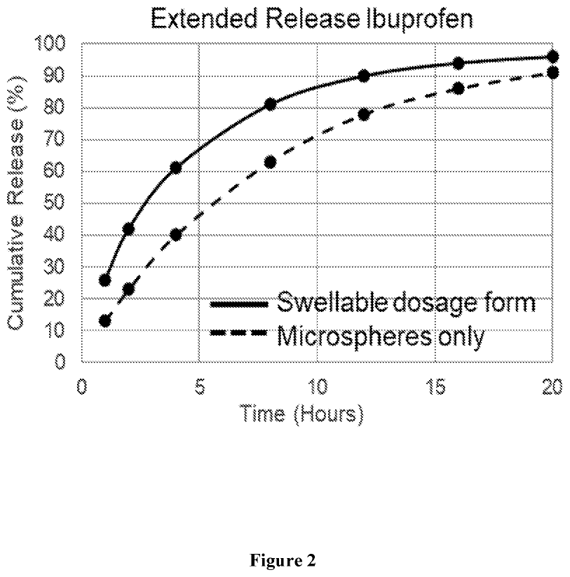

For dissolution testing, the pharmaceutical composition was dissolution tested using a USP Type II apparatus in 900 mL of pH 7.2 sodium phosphate buffer at 50 rpm (n=6), and compared to dissolution of the microsphere component only (see FIG. 2).

### Example 2. Preparation of Immediate-Release Cetirizine Tablet

To make the drug component (microspheres), a molten solution consisting of 45% carnauba wax, 45% glyceryl monostearate, 5% Eudragit® E-PO, and 5% cetirizine was made under stirring at 100° C. stirring at 100° C. The molten solution was then processed via a melt spray congeal process, which utilizes an accelerating co-flowing gas stream, combined with piezoelectric vibration. The resulting cooled powder exhibited a size distribution with a D[4,3]˜250 μm.

To make the swellable component, 57.6% SMCC Prosolv®, 28.8% gellan gum (HA), 7.2% mannitol, 4.3% calcium citrate, 0.6% D&C Red #7, and 1.0% magnesium stearate were high shear granulated.

Next, to make the pharmaceutical composition, the microsphere component was dry blended with the swellable component such that the microspheres constituted 30% of the dosage form weight, corresponding to a 10 mg cetirizine dose.

Finally, to make the tablet, the powder blend was compressed into a tablet using a 15 mm FFRE tooling at 350 psi.

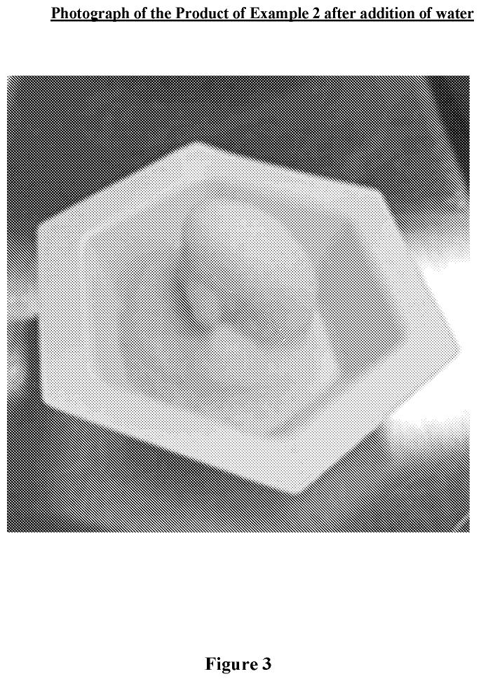

For swell testing of the dosage form, 7 mL of tap water was added to the dry tablet on a spoon to create the palatable matrix with a “soft serve” like appearance over a 43 second duration (n=10). A photograph of the product after the addition of water is displayed in FIG. 3.

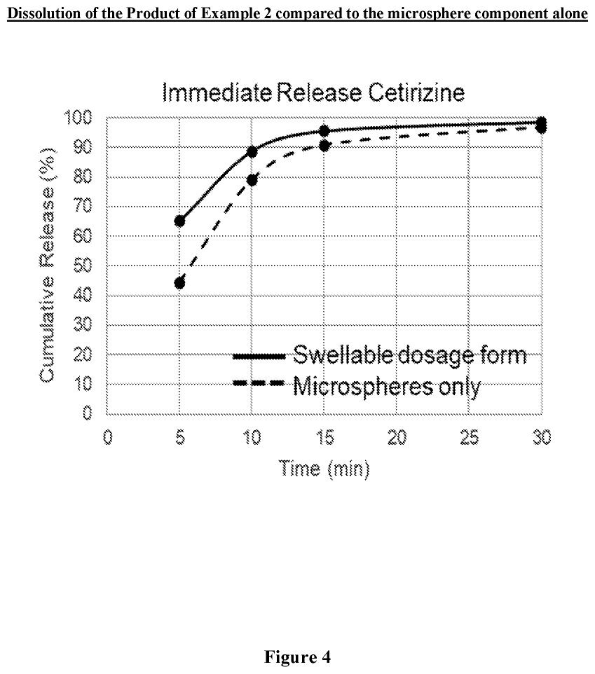

For dissolution testing, the tablet was dissolution tested using a USP Type II apparatus in 900 mL of 0.1N HCl at 50 rpm (n=6), and compared to dissolution of the microsphere component only (see FIG. 4).

### Example 3. Acetaminophen 160 mg Tablet with Berry flavor

Drug component preparation: Acetaminophen (APAP) microcapsules were prepared by a coacervation process with ethylcellulose in cyclohexane.

Pre-blend preparation: Calcium citrate tetrahydrate, citric acid, malic acid, color, flavor and sucralose were added to a 2 L blender and blended for 10 minutes at 20 rpm. The blend was then discharged and passed through a comil 045R screen at 1850 rpm.

Blend preparation: APAP microcapsules (coacervated Microcaps®), the pre-blend, SMCC, lactose and gellan gum were added to a 15 L blender and blended for 20 min at 20 rpm. Magnesium stearate was added and the mixture was blended for 5 minutes at 20 rpm.

Tabletting: The blend was tableted for a total tablet weight of 709 mg and diameter of 13 mm. Friability is 0.3%, hardness is 35N.

TABLE 1
The composition of the APAP 160 mg tablet with Berry flavor

| Ingredients | % w/w |
|---|---|
| APAP Microcaps | 24.0 |
| Gellan Gum (HA) | 20.0 |
| Silicified MCC, NF | 40.0 |
| Lactose Monohydrate, NF | 6.4 |
| Calcium citrate tetrahydrate | 3.0 |
| Citric Acid, Anhydrous USP | 1.2 |
| Wildberry Natural Flavor | 2.0 |
| D&C Red #7, Calcium Lake 17%-25% (31DA3707) | 0.4 |
| Malic Acid NF | 1.2 |
| Sucralose NF | 0.8 |
| Magnesium Stearate NF | 1.0 |

TABLE 2
Analytical attributes of APAP 160 mg tablet with Berry flavor

| Attribute | Specification | Results |
|---|---|---|
| Physical Description | Meets Spec | PASS |
| ID-Swell Test¹ | NMT 120 sec | 67 |
| ID-Inversion Test² | Remains in Container | PASS |
| Disintegration | FIO (sec) | <30 |
| Assay | 90-110 (%) | 101.4 |
| Moisture by LOD | FIO (%) | 4.2 |
| Content Uniformity | AV NMT 15 | 2.7 |

¹Tablet swelled into a semisolid gel within 120 seconds upon reconstitution in 5 ml water. A tablet with diameter of about 1 cm swelled into a tablet-shaped mass of about 2 cm
²Reconstituted product remained in the container in its entirety when inverted

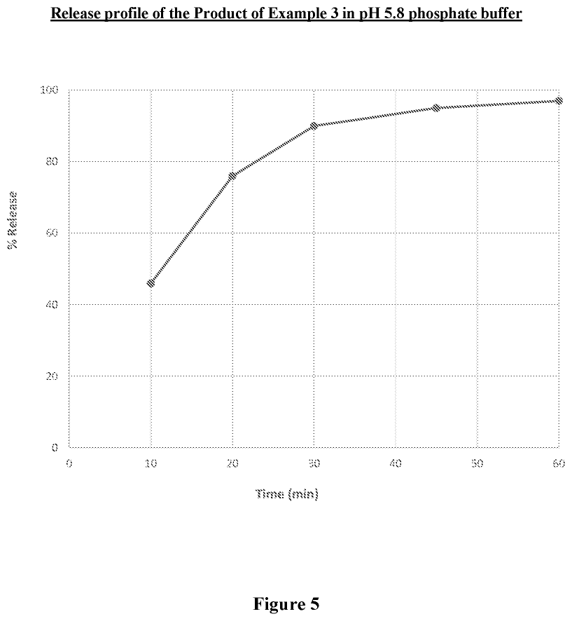

The release profile of APAP 160 mg tablet with Berry flavor was obtained in pH 5.8 phosphate buffer, 50 rpm. USP Spec: NLT 80% @ 30 min (see FIG. 5).

Stability data: APAP 160 mg tablet with Berry flavor were put on stability testing under long term conditions (25° C., 60% humidity) and under accelerated conditions (40° C., 75% humidity) in bottles without desiccant. The results are presented in Table 3.

TABLE 3
Stability data

| | | | Long Term | | | | Accelerated | | |
|---|---|---|---|---|---|---|---|---|---|
| | | | 1 Month | 3 Month | 6 Month | 9 Month | 1 Month | 3 Month | 6 Month |
| Attribute | Specification | Initial | 25°/60% | 25°/60% | 25°/60% | 25°/60% | 40°/75% | 40°/75% | 40°/75% |
| Physical | Meets Spec | PASS | PASS | PASS | PASS | PASS | PASS | PASS | PASS |
| Description | | | | | | | | | |
| ID - Swell | NMT 120 s | 67 | 69 | 63 | 70 | 72 | 65 | 76 | 85 |
| Test | | | | | | | | | |
| ID - | Remains in | PASS | PASS | PASS | PASS | PASS | PASS | PASS | 1 fail |
| Inversion | container | | | | | | | | |
| Assay | 90-110 (%) | 101.4 | 100.1 | 99.5 | 101.8 | 101.9 | 98.9 | 99.7 | 99.7 |
| Moisture by | FIO (%) | 4.2 | 4.8 | 4.2 | 3.9 | 4.0 | 4.3 | 4.3 | 3.8 |
| LOD | | | | | | | | | |
| Disintegration | FIO (s) | <30 | <30 | <30 | <30 | <30 | <30 | <30 | <30 |
| Tablet | FIO (N) | 35 | 37 | 37 | 38 | 38 | 43 | 40 | 37 |
| Hardness | | | | | | | | | |
| Tablet | NMT 2.0(%) | 0.3 | 0.3 | 0.2 | 0.2 | 0.4 | 0.2 | 0.2 | 0.4 |
| Friability | | | | | | | | | |
| Dissolution | NLT 80% @ | 90 | 90 | 86 | 94 | 94 | 93 | 88 | 92 |
| | 30 min | | | | | | | | |
| Impurities | | | | | | | | | |
| PAP: | NMT 0.15% | 0.0 | 0.0 | 0.0 | 0.0 | 0.0 | 0.0 | 0.0 | 0.0 |
| Unspecified: | NMT 0.15% | <LOD | <LOD | <LOD | <LOD | <LOD | LOD | <LOD | <LOD |
| Total: | NMT 0.6% | 0.0 | 0.0 | 0.031 | 0.0 | 0.173 | 0.0 | 0.028 | 0.023 |

Stability data: APAP 160 mg tablet with Berry flavor were put on stability testing under long term conditions (25° C., 60% humidity) and under accelerated conditions (40° C., 75% humidity) in bottles with desiccant. The results are presented in Table 4.

TABLE 4
Stability data

| | | | Long Term | | | | Accelerated | | |
|---|---|---|---|---|---|---|---|---|---|
| | | | 1 Month | 3 Month | 6 Month | 9 Month | 1 Month | 3 Month | 6 Month |
| Attribute | Specification | Initial | 25°/60% | 25°/60% | 25°/60% | 25°/60% | 40°/75% | 40°/75% | 40°/75% |
| Physical | Meets Spec | PASS | PASS | PASS | PASS | PASS | PASS | PASS | PASS |
| Description | | | | | | | | | |
| ID - Swell | NMT 120 s | 67 | 67 | 64 | 67 | 70 | 65 | 80 | 84 |
| Test | | | | | | | | | |
| ID - | Remains in | PASS | PASS | PASS | PASS | PASS | PASS | PASS | PASS |
| Inversion | container | | | | | | | | |
| Assay | 90-110 (%) | 101.4 | 99.9 | 100.0 | 99.7 | 101.8 | 100.9 | 100.4 | 100.6 |
| Moisture by | FIO (%) | 4.2 | 4.3 | 4.7 | 4.0 | 3.8 | 4.4 | 4.5 | 3.6 |
| LOD | | | | | | | | | |
| Disintegration | FIO (s) | <30 | <30 | <30 | <30 | <30 | <30 | <30 | <30 |
| Tablet | FIO (N) | 35 | 36 | 37 | 35 | 36 | 43 | 40 | 37 |
| Hardness | | | | | | | | | |
| Tablet | NMT 2.0(%) | 0.3 | 0.2 | 0.2 | 0.3 | 0.3 | 0.2 | 0.2 | 0.4 |
| Friability | | | | | | | | | |
| Dissolution | NLT 80% @ | 90 | 93 | 89 | 94 | 93 | 94 | 89 | 89 |
| | 30 min | | | | | | | | |
| Impurities | | | | | | | | | |
| PAP: | NMT 0.15% | 0.0 | 0.0 | 0.0 | 0.0 | 0.0 | 0.0 | 0.0 | 0.0 |
| Unspecified: | NMT 0.15% | <LOD | <LOD | <LOD | <LOD | <LOD | <LOD | <LOD | <LOD |
| Total: | NMT 0.6% | 0.0 | 0.0 | 0.019 | 0.0 | 0.186 | 0.0 | 0.028 | 0.020 |

### Example 4. Acetaminophen 160 mg Tablet with Cherry Flavor

Drug component preparation: Acetaminophen (APAP) microcapsules were prepared by coacervation process with ethylcellulose in cyclohexane.

Pre-blend preparation: Calcium citrate tetrahydrate, citric acid, color, flavor and sucralose were added to a 3 L blender and blended for 10 minutes at 20 rpm. The blend was then discharged and passed through a comil 045R screen at 1850 rpm.

Blend preparation: APAP microcapsules (coacervated Microcaps), the pre-blend, SMCC, lactose and gellan gum were added to a 15 L blender and blended for 20 min at 20 rpm. Magnesium stearate was then added and the mixture was blended for 5 minutes at 20 rpm.

Tabletting: The blend was tableted for a total tablet weight of 709 mg and diameter of 13 mm. Friability is 0.3% and hardness is 34 N.

TABLE 5
Composition of the APAP 160 mg tablet with Cherry flavor

| Ingredients | % w/w |
|---|---|
| APAP Microcaps | 24.0 |
| Gellan Gum (HA) | 20.0 |
| Silicified MCC, NF | 40.0 |
| Lactose Monohydrate, NF | 7.6 |
| Calcium Citrate tetrahydrate | 3.0 |
| Citric Acid, Anhydrous USP | 1.2 |
| Artificial Cherry Flavor | 2.0 |
| D&C Red #7, Calcium Lake 17%-25% (31DA3707) | 0.4 |
| Sucralose NF | 0.8 |
| Magnesium Stearate NF | 1.0 |

TABLE 6
Analytical Attributes of APAP 160 mg tablet with Cherry flavor

| Attribute | Specification | Results |
|---|---|---|
| Physical Description | Meets Spec | PASS |
| ID-Swell Test¹ | NMT 120 sec | 55 |
| ID-Inversion Test² | Remains in Container | PASS |
| Disintegration | FIO (sec) | <30 |
| Assay | 90-110 (%) | 101.1 |
| Moisture by LANDLORD | FIO (%) | 4.4 |
| Content Uniformity | AV NMT 15 | 1.7 |

¹Tablet swelled into a semisolid gel within 120 seconds upon reconstitution in 5 ml water
²Reconstituted product remained in the container in its entirety when inverted

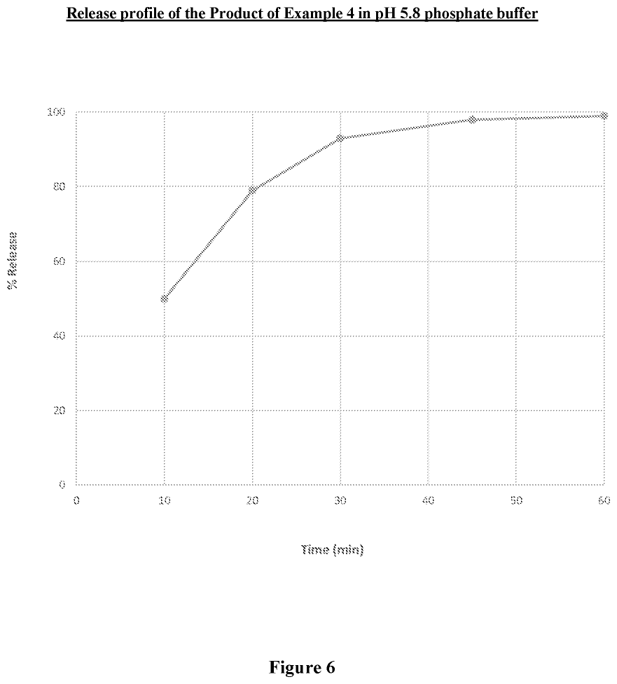

The release profile of APAP 160 mg tablet with Cherry flavor was obtained in pH 5.8 phosphate buffer, 50 rpm. USP Spec: NLT 80% @ 30 min (see FIG. 6).

Stability data: APAP 160 mg tablet with Cherry flavor were put on stability testing under long term conditions (25° C., 60% humidity) and under accelerated conditions (40° C., 75% humidity) in bottles without desiccant. The results are presented in Table 7.

TABLE 7
Stability data

| | | | Long Term | | | | Accelerated | | |
|---|---|---|---|---|---|---|---|---|---|
| | | | 1 Month | 3 Month | 6 Month | 9 Month | 1 Month | 3 Month | 6 Month |
| Attribute | Specification | Initial | 25°/60% | 25°/60% | 25°/60% | 25°/60% | 40°/75% | 40°/75% | 40°/75% |
| Physical | Meets Spec | PASS | PASS | PASS | PASS | PASS | PASS | PASS | PASS |
| Description | | | | | | | | | |
| ID - Swell | NMT 120 s | 55 | 54 | 47 | 51 | 52 | 64 | 63 | 73 |
| Test | | | | | | | | | |
| ID - | Remains in | PASS | PASS | PASS | PASS | PASS | PASS | PASS | PASS |
| Inversion | container | | | | | | | | |
| Assay | 90-110 (%) | 101.1 | 100.0 | 97.4 | 99.5 | 101.7 | 100.1 | 98.9 | 101.3 |
| Moisture by | FIO (%) | 4.4 | 4.5 | 4.7 | 4.2 | 4.2 | 4.7 | 4.5 | 4.3 |
| LOD | | | | | | | | | |
| Disintegration | FIO (s) | <30 | <30 | <30 | <30 | <30 | <30 | <30 | <30 |
| Tablet | FIO (N) | 34 | 36 | 35 | 34 | 36 | 39 | 38 | 34 |
| Hardness | | | | | | | | | |
| Tablet | NMT 2.0(%) | 0.3 | 0.2 | 0.2 | 0.3 | 0.2 | 0.2 | 0.2 | 0.4 |
| Friability | | | | | | | | | |
| Dissolution | NLT 80% @ | 93 | 94 | 89 | 95 | 96 | 95 | 88 | 89 |
| | 30 min | | | | | | | | |
| Impurities | | | | | | | | | |
| PAP: | NMT 0.15% | 0.0 | 0.0 | 0.0 | 0.0 | 0.0 | 0.0 | 0.0 | 0.0 |
| Unspecified: | NMT 0.15% | <LOD | <LOD | <LOD | <LOD | <LOD | <LOD | <LOD | <LOD |
| Total: | NMT 0.6% | 0.0 | 0.0 | 0.027 | 0.037 | 0.204 | 0.0 | 0.016 | 0.021 |

Stability data: APAP 160 mg tablet with Cherry flavor were put on stability testing under long term conditions (25° C., 60% humidity) and under accelerated conditions (40° C., 75% humidity) conditions in bottles with desiccant. The results are presented in Table 8.

TABLE 8
Stability data

| | | | Long Term | | | | Accelerated | | |
|---|---|---|---|---|---|---|---|---|---|
| | | | 1 Month | 3 Month | 6 Month | | 1 Month | 3 Month | 6 Month |
| Attribute | Specification | Initial | 25°/60% | 25°/60% | 25°/60% | 9 Month | 40°/75% | 40°/75% | 40°/75% |
| Physical | Meets Spec | PASS | PASS | PASS | PASS | PASS | PASS | PASS | PASS |
| Description | | | | | | | | | |
| ID - Swell | NMT 120 s | 55 | 53 | 50 | 49 | 53 | 63 | 61 | 71 |
| Test | | | | | | | | | |
| ID - | Remains in | PASS | PASS | PASS | PASS | PASS | PASS | PASS | PASS |
| Inversion | container | | | | | | | | |
| Assay | 90-110 (%) | 101.1 | 100.0 | 100.2 | 100.1 | 100.8 | 99.3 | 98.0 | 100.3 |
| Moisture by | FIO (%) | 4.4 | 4.4 | 4.8 | 4.2 | 3.8 | 4.5 | 4.3 | 4.0 |
| LOD | | | | | | | | | |
| Disintegration | FIO (s) | <30 | <30 | <30 | <30 | <30 | <30 | <30 | <30 |
| Tablet | FIO (N) | 34 | 35 | 34 | 35 | 36 | 40 | 37 | 35 |
| Hardness | | | | | | | | | |
| Tablet | NMT 2.0(%) | 0.3 | 0.2 | 0.2 | 0.2 | 0.2 | 0.2 | 0.2 | 0.4 |
| Friability | | | | | | | | | |
| Dissolution | NLT 80% @ | 93 | 93 | 90 | 94 | 94 | 95 | 89 | 94 |
| | 30 min | | | | | | | | |
| Impurities | | | | | | | | | |
| PAP: | NMT 0.15% | 0.0 | 0.0 | 0.0 | 0.0 | 0.0 | 0.0 | 0.0 | 0.0 |
| Unspecified: | NMT 0.15% | <LOD | <LOD | <LOD | <LOD | <LOD | <LOD | <LOD | <LOD |
| Total: | NMT 0.6% | 0.0 | 0.0 | 0.025 | 0.037 | 0.198 | 0.0 | 0.030 | 0.035 |

### Example 5. Swelling Testing on Acetaminophen 160 mg Tablet

Drug component preparation: Acetaminophen (APAP) microcapsules were prepared by a coacervation process with ethylcellulose in cyclohexane.

Pre-blend preparation: Calcium citrate tetrahydrate, citric acid, color and sucralose were added to a 2 L blender and blended for 10 minutes at 20 rpm. The blend was then discharged and passed through a comil 045R screen at 1850 rpm.

Blend preparation: APAP microcapsules (coacervated Microcaps), the pre-blend, SMCC, lactose and gellan gum were added to a 15 L blender and blended for 20 min at 20 rpm. Magnesium stearate was added and mixture was blended for 5 minutes at 20 rpm.

Tabletting: The blend was tableted for a total tablet weight of 473 mg and diameter of 12 mm.

TABLE 9
Composition of the APAP 160 mg tablet

| | % w/w | % w/w |
|---|---|---|
| | (contains | (contains |
| Ingredients | 2% salt) | 3% salt) |
| APAP Microcaps | 36.0 | 36.0 |
| Gellan Gum (HA) | 30.0 | 25.0 |
| Silicified MCC, NF | 22.0 | 22.0 |
| Lactose Monohydrate, NF | 6.4 | 8.6 |
| Calcium citrate tetrahydrate | 2.0 | 3.0 |
| Citric Acid, Anhydrous USP | 1.2 | 1.2 |
| D&C Red #7, Calcium Lake | 0.4 | 0.4 |
| 17%-25% (31DA3707) | | |
| Sucralose NF | 0.8 | 0.8 |
| Magnesium Stearate NF | 1.0 | 1.0 |

Swelling was performed with a reconstitution volume of 4 ml of water. The swelling time for compositions comprising 2% salts varied from 180 seconds to 28 seconds depending on the water type used for swelling (hard, type, purified water). When the formulations incorporated salt to 3% w/w, the swell times varied from 37 to 33 seconds on average independent of the water type. These formulations swelled completely also in the most inner core parts and did not have any internal gummy-like center.

### Example 6. Swelling Testing on Acetaminophen 160 mg Tablet

Drug component preparation: Acetaminophen (APAP) microcapsules were prepared by a coacervation process with ethylcellulose in cyclohexane.

Pre-blend preparation: Calcium citrate tetrahydrate, citric acid, color, flavor and sucralose were added to a 2 L blender and blended for 10 minutes at 20 rpm. The blend was then discharged and passed through a comil 045R screen at 1850 rpm.

Blend preparation: APAP microcapsules (coacervated Microcaps), the pre-blend, SMCC, lactose and gellan gum were added to a 15 L blender and blended for 20 minutes at 20 rpm. Magnesium stearate was added and mixture was blended for 5 minutes at 20 rpm.

Tabletting: The blend was tableted for a total tablet weight of 567 mg and diameter of 12 mm.

TABLE 10
Composition of the APAP tablet with different flavors and different amount of flavor

| | | | | | | % w/w |
|---|---|---|---|---|---|---|
| | % w/w | % w/w | % w/w | % w/w | % w/w | 2% Flavor |
| Ingredients | Flavor 1 | Flavor 2 | no Flavor | 2% Flavor | Flavor 3 | sieved |
| APAP Microcaps | 30.0 | 30.0 | 30.0 | 30.0 | 30.0 | 30.0 |
| Gellan Gum (HA) | 25.0 | 25.0 | 25.0 | 25.0 | 25.0 | 25.0 |
| Silicified MCC, NF | 25.0 | 25.0 | 25.0 | 25.0 | 25.0 | 25.0 |
| Lactose Monohydrate, NF | 8.3 | 8.3 | 13.3 | 11.3 | 8.3 | 11.3 |
| Calcium citrate tetrahydrate | 2.0 | 2.0 | 2.0 | 2.0 | 2.0 | 2.0 |
| Citric Acid, Anhydrous USP | 1.5 | 1.5 | 1.5 | 1.5 | 1.5 | 1.5 |
| Berry Flavor 1 | 5.0 | | | 2.0 | | 2.0 |
| Berry Flavor 2 | | 5.0 | | | | |
| Berry Flavor 3 | | | | | 5.0 | |
| D&C Red #7, Calcium Lake | 0.4 | 0.4 | 0.4 | 0.4 | 0.4 | 0.4 |
| 17%-25% (31DA3707) | | | | | | |
| Sucralose NF | 0.8 | 0.8 | 0.8 | 0.8 | 0.8 | 0.8 |

Swelling was performed with a reconstitution volume of 4 ml of water. The formulation with 2% flavor had a swelling time of 33 seconds in purified water and 32 seconds in tap water. However, it contained an unswelled internal core. The formulation with 2% flavor sieved (de-lumping/sieving step aids the dispersion of the flavor within the blend matrix) had a swelling time of 31 seconds in purified water and 30 seconds in tap water. It contained no un-swelled core.

### Example 7. Swelling Testing on Acetaminophen 160 mg Tablet

Drug component preparation: Acetaminophen microcapsules were prepared by a coacervation process with ethylcellulose in cyclohexane.

Pre-blend preparation: Calcium citrate tetrahydrate, citric acid, color and sucralose were added to a 2 L blender and blended for 10 minutes at 20 rpm. The blend was then discharged and passed through a comil 045R screen at 1850 rpm.

Blend Preparation: APAP microcapsules (coacervated Microcaps), the pre-blend, SMCC, lactose and gellan gum were added to a 15 L blender and blended for 20 minutes at 20 rpm. Magnesium stearate was added and the mixture was blended for 5 minutes at 20 rpm

Tabletting: The blend was tableted as pink, round, lozenge shaped tablets.

TABLE 11
Composition of the APAP 160 mg tablets

| | % w/w | % w/w |
|---|---|---|
| Ingredients | (contains mannitol) | (contains lactose) |
| APAP Microcaps | 30.25 | 24.0 |
| Gellan Gum | 25.0 | 20.0 |
| Silicified MCC, NF | 25.0 | 40.0 |
| Mannitol | 8.15 | — |
| Lactose Monohydrate, NF | — | 6.4 |
| Calcium citrate tetrahydrate | 3.0 | 3.0 |
| Citric Acid, Anhydrous USP | 1.2 | 1.2 |
| Malic acid | 1.2 | 1.2 |
| D&C Red #7, Calcium Lake | 0.4 | 0.4 |
| 17%-25% (31DA3707) | | |
| Berry Flavor | 4.0 | 2.0 |
| Sucralose NF | 0.8 | 0.8 |
| Magnesium Stearate NF | 1.0 | 1.0 |

Swelling was performed with a reconstitution volume of 4 ml of water. Tablets with mannitol had a total tablet weight of 565 mg and tablets with lactose had a total tablet weight of 709 mg.

Mannitol tablet: as hardness was increased (from 14N to 25N) to improve friability (from >2.0% to 1.0%) the swelled time was extended and the swelled tablet had an observed un-swelled core. The swelling was 37 seconds for the tablet with low hardness (14N) and high friability (>2%) and was >120 seconds for the tablet with high hardness (25N) and low friability (1.0%).

Lactose tablet: high hardness (35N) was achieved with less compression force. The tablet with lactose had a friability of 3% and properly swelled in both purified (51 seconds) and tap water (67 seconds). It did not have any internal gummy-like center (no un-swelled core).

### Example 8. Uncoated Placebo Minitabs+Minitabs of Swellable Component

### Step 1: Preparation of Uncoated Placebo Minitabs (2.0 mm)

A L-IBC blender was charged with lactose monohydrate (69.5 parts) and silicified microcrystalline cellulose (SMCC 90) (29.5) parts and blended for 15 minutes at 10 rpm. Sodium stearyl fumarate (1 part) was sieved through a 35 mesh sieve and then added to the blender (containing the lactose and the SMCC) and further blended for 5 minutes producing a homogenous blend for compression (batch size: 1 kg). A rotary tablet press, Manesty Betapress, equipped with a minitablet tool set (16, each 2 mm in diameter) configured for minitablet target weight of 5.5 mg, was set up with the following compression parameters: fill depth setting: 2 mm; Compression force setting: less than or equal to 1.3 kN (main compression: about 2.5 mm); Pre-compression setting: 0.12 kN (Pre compression: about 8 mm); Force feeder speed setting: 0 Turret rpm: 35; Average weight of Minitabs tested: 7.1 mg/Minitab and hardness of 14 N.

### Step 2: Preparation of Swellable Component in Form of Minitabs (2.0 mm)

A L-IBC blender was charged with Gellan Gum (Kelcogel CG-HA) (20.0 parts), silicified microcrystalline cellulose (SMCC 90) (15.0 parts), Mannitol Granulation (59.8 parts), Citric Acid (1.2 parts), and Calcium Citrate Tetrahydrate (3.0 parts) and blended for 15 minutes at 10 rpm. Magnesium Stearate NF (1.0 part) was added to the blender (containing Gellan Gum, SMCC90, Mannitol Granulation, Citric Acid and Calcium Citrate Tetrahydrate) and further blended for 5 minutes producing a homogenous blend for compression (batch size: 1 kg). A rotary tablet press, Manesty Betapress, equipped with a minitablet tool set (16, each 2 mm in diameter) configured for minitablet target weight of 10.0 mg, was set up with the following compression parameters: fill depth: setting: 2 mm; Compression force setting: less than or equal to 1.3 kN (main compression: about 2.5 mm); Pre-compression setting: 0.12 kN (Pre compression: about 8 mm); Force feeder speed setting: 0 Turret rpm: 35.

### Step 3: Combining the Products of Steps 1 and 2

Uncoated placebo Minitabs prepared in Step 1 were added to a spoon. Then Minitabs of swellable component prepared in Step 2 were added to the same spoon. Water was added to the spoon causing swelling of the components. A resulting homogeneous gel-like preparation was formed in a few minutes.

### Example 9. Coated Placebo Minitabs+Minitabs of Swellable Component

### Step 1: Preparation of Coated Placebo Minitabs (2.2 mm)

Minitabs were prepared as described in Example 8, Step 1. These Minitab cores (1,818.2 g) were provided with a stabilizing coating comprising an OPADRY II white coating (363.6 g) dissolved/dispersed in 2,060.4 g of USP water in a Glatt GPCG-3 equipped with a 6″ Wurster insert, peristaltic pump and 0.8 mm nozzle tip size for a spray rate of 6 mL/minute ramp to 12 mL/min, Air distribution plate ‘D’ and 100 mesh product support screen, and dedicated filter bag at the following parameters: Inlet temperature setting—61° C.; Process air volume—70 cfm; Atomization air—1.0 bar; Target product temperature: 43-47° C. These coated minitabs were then compressed as described in Example 8, Step 1.

### Step 2: Combining the Product of Step 1 Above and Product of Example 8, Step 2

Coated placebo Minitabs prepared in Step 1 were added to a spoon. Then minitabs of swellable component prepared in Example 8, Step 2 were added to the same spoon. Water was added to the spoon causing swelling of the components. A resulting homogeneous gel-like preparation was formed in few minutes.

### Example 10. Swellable Placebo Tablet Containing Xylitol

Xylitol was passed through a Comill 032R (0.032 inch) screen at 1850 rpm. Color, flavor and sucralose were combined with 100 g milled xylitol and then mixed for not less than 2 minutes. The combined color, flavor, sucralose and xylitol were passed through the Comill. Kelcogel, the milled combined color, flavor, sucralose and xylitol, and an additional 100 g milled Xylitol, were added to the bin in this order and then blended for 30 min at 15 rpm. 300 g of the blend was removed. Sodium stearyl fumarate was added to the blend and mixed for 10 min at 15 rpm. The blend was tableted using a 14 mm round, dimple tooling at a tablet weight of 600 mg. Friability of the tablets was measured according to US Pharmacopeial Convention <1216> and found to be above 1% which is not acceptable for a tablet per FDA guidance. It is also known that friability values above 1% are not considered suitable robust for packaging and shipping. The tablets exhibited a swell time of 65-75 seconds.

TABLE 12
Composition of swellable placebo tablet containing Xylitol

| Ingredients | % w/w |
|---|---|
| Gellan Gum (HA) Kelcogel CG-HA | 19.8 |
| Xylitol 10-40 mesh | 69.4 |
| Cherry Flavor | 8.7 |
| D&C Red #7 | 0.4 |
| Sucralose NF | 1.0 |
| Sodium Stearyl Fumarate | 0.7 |

### Example 11. Swellable Placebo Tablet Containing Xylitol and Silicified Microcrystalline Cellulose (SMCC) at 5, 10, 20, 30, and 40% w/w

Prosolv SMCC90 was incorporated into the xylitol blend described in Example 10 at 5, 10, 20, 30, and 40% w/w and the blends tableted with 14 mm, round dimple shaped tooling with target weight of 600 mg using a carver press at a force of 300 psi. Tablet hardness was observed to increase from 13 N to 24 N as the percentage of SMCC increased in the formulation and the corresponding % of xylitol decreased. Swelling time decreased from 63 seconds for the 5% SMCC formulation to 33 seconds for the 40% SMCC formulation.

TABLE 13
Composition of swellable placebo tablet containing Xylitol and SMCC

| | % w/w | | | | |
|---|---|---|---|---|---|
| | 5% | 10% | 20% | 30% | 40% |
| Ingredients | SMCC | SMCC | SMCC | SMCC | SMCC |
| Gellan Gum (HA) | 19.8 | 19.8 | 19.8 | 19.8 | 19.8 |
| Kelcogel CG-HA | | | | | |
| Xylitol 10-40 mesh | 64.4 | 59.4 | 49.4 | 39.4 | 29.4 |
| SMCC90 Prosolv | 5.0 | 10.0 | 20.0 | 30.0 | 40.0 |
| Cherry Flavor | 8.7 | 8.7 | 8.7 | 8.7 | 8.7 |
| D&C Red #7 | 0.4 | 0.4 | 0.4 | 0.4 | 0.4 |
| Sucralose NF | 1.0 | 1.0 | 1.0 | 1.0 | 1.0 |
| Sodium Stearyl Fumarate | 0.7 | 0.7 | 0.7 | 0.7 | 0.7 |
| Tablet Hardness (N) | 13 | 15 | 19 | 23 | 24 |
| Swell Time (seconds) | 62 | 55 | 48 | 38 | 33 |

### Example 12. Swellable Placebo Tablet Containing Xylitol and Silicified Microcrystalline Cellulose (SMCC) without Flavor or Color

SMCC, Xylitol, Kelcogel and sucralose were mixed together for not less than 5 minutes. Sodium stearyl fumarate was added to the bin and blended for not less than 2 minutes. Tablets were prepared using a carver press and 14 mm lozenge tooling at 600 PSI. The prepared tablet has a hardness of 35-45N. Friability of the tablet is about 0.7%-0.9%.

TABLE 14
Composition of swellable placebo tablet
containing Xylitol and SMCC (40%)

| Ingredients | % w/w |
|---|---|
| Gellan Gum (HA) Kelcogel CG-HA | 20.0 |
| Xylitol 10-40 mesh | 38.3 |
| SMCC90 Prosolv | 40.0 |
| Sucralose NF | 0.7 |
| Sodium Stearyl Fumarate | 1.0 |

### Example 13. Swellable Placebo Tablet Containing Xylitol and Silicified Microcrystalline Cellulose (SMCC)

Xylitol was passed through a Comill 032R (0.032 inch) round screen. Color, flavor and sucralose were combined with 100 g milled xylitol and then mixed for not less than 2 minutes. The combined color, flavor, sucralose and xylitol were passed through the Comill. Approximately half of the SMCC, half of the xylitol, gellan gum, the milled color, flavor, sucralose and the remaining half of the SMCC and xylitol were added to the bin in this order and then blended for 30 min at 15 rpm. Sodium stearyl fumarate was added to the bin and blended for 15 minutes at 15 rpm. The blend was tableted with a Fetti 52i Tablet Press with 4 stations of tooling of 14 mm, round, lozenge tooling with target weight of 600 mg and with the following compression parameters: 2.32 (main compression), Pre-compression setting about 0.64 kN; Force feeder speed setting: 40 Turret rpm: 5. Prepared tablets had a hardness of 28-48 N. Friability of the tablets is about 0.6%-0.9%.

TABLE 15
Composition of swellable placebo tablet
containing Xylitol and SMCC (39.6%)

| Ingredients | % w/w |
|---|---|
| Gellan Gum (HA) Kelcogel CG-HA | 19.8 |
| Xylitol 10-40 mesh | 31.8 |
| SMCC90 Prosolv | 39.6 |
| Cherry Flavor | 6.7 |
| D&C Red #7 | 0.4 |
| Sucralose NF | 0.7 |
| Sodium Stearyl Fumarate | 1.1 |

### Example 14. Swellable Placebo Tablet Containing Silicified Microcrystalline Cellulose (SMCC) and Either Mannitol (Samples A and B) or Lactose (Samples C and D) Instead of Xylitol

In this preparation xylitol present in previous examples was replaced by another ingredient, namely by mannitol (Mannogem 2080 and Partek M200) or lactose (Capsulac 60 and Lactose Monohydrate) and this blend was tableted. Formulations comprising xylitol, even with the presence of a compression aid of SMCC, exhibited higher than desired tablet friability. Mannitol and lactose were here evaluated as alternative hydrophilic agents to xylitol. It has been found that these agents improve the robustness of the formulation. Tablets manufactured were tested for swelling time and tablet hardness.

All ingredients, with the exception of sodium stearyl fumarate and half of SMCC were combined and then mixed for not less than 2 minutes. The combined color, flavor, sucralose, ½ SMCC and either mannitol or lactose were passed through the Comill. Approximately half of the SMCC and sodium stearyl fumarate were added to the bin and blended for 2 min at 15 rpm. The blends were compressed with a Carver Press with 14 mm, round, lozenge tooling to 300 psi.

TABLE 16
Composition of swellable placebo tablet containing Mannogem
2028 and SMCC (39.4%)-Tablet A

| Ingredients | % w/w |
|---|---|
| Gellan Gum (HA) Kelcogel CG-HA | 20.0 |
| Mannogem 2028 (Sample A) | 31.8 |
| SMCC90 Prosolv | 39.4 |
| Cherry Flavor | 6.7 |
| D&C Red #7 | 0.4 |
| Sucralose NF | 0.7 |
| Sodium Stearyl Fumarate | 1.2 |

TABLE 17
Composition of swellable placebo tablet containing
Partek M200 and SMCC (39.4%)-Tablet B

| Ingredients | % w/w |
|---|---|
| Gellan Gum (HA) Kelcogel CG-HA | 20.0 |
| Partek M200 (Sample B) | 31.8 |
| SMCC90 Prosolv | 39.4 |
| Cherry Flavor | 6.7 |
| D&C Red #7 | 0.4 |
| Sucralose NF | 0.7 |
| Sodium Stearyl Fumarate | 1.2 |

TABLE 18
Composition of swellable placebo tablet containing
Capsulac 60 and SMCC (39.4%)-Tablet C

| Ingredients | % w/w |
|---|---|
| Gellan Gum (HA) Kelcogel CG-HA | 20.0 |
| Capsulac 60 (Sample C) | 31.8 |
| SMCC90 Prosolv | 39.4 |
| Cherry Flavor | 6.7 |
| D&C Red #7 | 0.4 |
| Sucralose NF | 0.7 |
| Sodium Stearyl Fumarate | 1.2 |

TABLE 19
Composition of swellable placebo tablet containing Lactose
mono and SMCC (39.4%)-Tablet D

| Ingredients | % w/w |
|---|---|
| Gellan Gum (HA) Kelcogel CG-HA | 20.0 |
| Lactose mono (Sample D) | 31.8 |
| SMCC90 Prosolv | 39.4 |
| Cherry Flavor | 6.7 |
| D&C Red #7 | 0.4 |
| Sucralose NF | 0.7 |
| Sodium Stearyl Fumarate | 1.2 |

The four tablets (Tablets A-D, including Samples A-D) produced according to Example 14, containing compositions described in Tables 15-18, were tested for water swelling and tablet hardness. One single tablet was placed into 1 weigh boat, then 5 ml of tap water was added and a timer started to measure the duration of time, in seconds) needed by the tablet to fully absorb water. This test was repeated six times. Table 20 describes the water swelling results obtained and tablet hardness.

TABLE 20

| Sample | Water Swelling (in seconds) | | | | | | Hardness (N) |
|---|---|---|---|---|---|---|---|
| A | 21 | 22 | 23 | 22 | 21 | 21 | 17 |
| B | 19 | 21 | 21 | 22 | 22 | 21 | 20 |
| C | 19 | 19 | 18 | 18 | 18 | 18 | 22 |
| D | 21 | 25 | 26 | 25 | 24 | 25 | 23 |

### Example 15. Swellable Placebo Tablet Containing Silicified Microcrystalline Cellulose (SMCC) and Lactose Instead of Xylitol

100 g of SMCC was added to color, flavor and sucralose and mixed for not less than 2 minutes and then is passed through a 100 mesh screen. Capsulac 60 (alpha lactose monohydrate with particle size distribution <100 μm NMT 10%, <250 μm 40-70%, <400 μm NLT 90%, <630 NMT 97%), Kelcogel, citric acid, the above blended SMCC, color, flavor and sucralose, and 100 g SMCC were added in this order to the bin and then blended for 30 min at 15 rpm. Sodium stearyl fumarate was added to the bin and blended for 15 min at 15 rpm. The blend was tableted with a Fetti 52i Tablet Press with 4 stations of tooling of 14 mm, round, lozenge tooling with target weight of 600 mg and with the following compression parameters: main compression setting of about 2.3 mm (˜14 kN), Pre-compression setting about 4.3 mm (˜0.7 Kn), Force feeder speed setting: 40 rpm, Turret setting: 5 rpm. Prepared tablets have a hardness value of from 34-53 N and thickness of approximately 3.6 mm. Friability of the tablets is between 0.2% and 0.5%.

TABLE 21
Composition of swellable placebo tablet
containing Lactose and SMCC (43.1%)

| Ingredients | % w/w |
|---|---|
| Gellan Gum (HA) Kelcogel CG-HA | 19.8 |
| Capsulac 60 | 31.6 |
| SMCC90 Prosolv | 43.1 |
| Cherry Flavor | 2.0 |
| D&C Red #7 | 0.4 |
| Sucralose NF | 0.8 |
| Citric acid | 1.2 |
| Sodium Stearyl Fumarate | 1.0 |

1. A solid pharmaceutical product comprising a drug-containing component and a swellable component, wherein the product is fully converted to a semi-solid form within about 2 minutes following the addition of water, without applying shear forces or other mixing forces.

2. The product according to claim 1, wherein the drug-containing component comprises a plurality of drug microparticles.

3. The product according to claim 2, wherein the drug microparticles have an average particle diameter of from about 50 μm to about 300 μm.

4. The product according to claim 2 or claim 3, wherein the drug microparticles are selected from drug microspheres and drug microcapsules.

5. The product according to any preceding claim, wherein the drug-containing component is a powder.

6. The product according to any preceding claim, wherein the swellable component consists of granules.

7. The product according to any preceding claim, wherein the drug-containing component and the swellable component are prepared separately and then dry blended as discrete solids.

8. The product according to claim 1, wherein the drug-containing component comprises a plurality of drug mini-tablets.

9. The product according to claim 1, wherein the swellable component comprises a plurality of swellable mini-tablets.

10. The product according to claim 8, wherein the drug mini-tablets have an average diameter of from about 1 mm to about 2 mm.

11. The product according to claim 9, wherein the swellable mini-tablets have an average diameter of from about 1 mm to about 2 mm.

12. The product according to claim 1, wherein the drug-containing component comprises a plurality of drug mini-tablets and the swellable component comprises a plurality of swellable mini-tablets.

13. The product according to claim 12, wherein the drug mini-tablets and the swellable mini-tablets each have an average diameter of from about 1 mm to about 2 mm.

14. The product according to claim 12 or claim 13, wherein the drug mini-tablets and the swellable mini-tablets are prepared separately and then dry blended as discrete solids.

15. The product according to any preceding claim, wherein the product is in the form of a unit dose powder or granulate.

16. The product according to any preceding claim, wherein the product is fully converted to a semi-solid form within about 30 seconds following the addition of water.

17. The product according to any one of claims 1-6, wherein the product is in the form of a unit dose tablet.

18. The product according to claim 17, wherein the tablet friability is 1% or less.

19. The product according to claim 17 or claim 18, wherein the product is fully converted to a semi-solid form within about 90 seconds following the addition of water.

20. The product according to claim 17 or claim 18, wherein the product is fully converted to a semi-solid form within about 45 seconds following the addition of water.

21. The product according to any preceding claim, wherein the swellable component comprises at least one water-swellable hydrophilic polymer.

22. The product according to claim 21, wherein the swellable component comprises about 20% to about 80% of the at least one water-swellable hydrophilic polymer.

23. The product according to claim 21 or 22, wherein a water-swellable hydrophilic polymer is a gellan gum.

24. The product according to claim 21 or claim 22, wherein a swellable hydrophilic polymer is gellan gum-high acyl.

25. The product according to any one of claims 21-24, wherein the swellable component further comprises one or more hydrophilic agents.

26. The product according to claim 25, wherein the hydrophilic agent is an osmogen.

27. The product according to claim 26, wherein the osmogen is selected from the group consisting of magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, inulin, instant sugar, citric acid, succinic acid, tartaric acid, and mixtures thereof.

28. A tablet comprising a drug-containing component and a swellable component, wherein the tablet is fully converted to a semi-solid form within about 2 minutes following the addition of water, without applying shear forces or other mixing forces.

29. The tablet according to claim 28, wherein the tablet friability is 1% or less.

30. The tablet according to claim 28 or claim 29, wherein the product is fully converted to a semi-solid form within about 90 seconds following the addition of water.

31. The tablet according to claim 28 or claim 29, wherein the product is fully converted to a semi-solid form within about 45 seconds following the addition of water.

32. The tablet according to any one of claims 28-31, wherein the drug-containing component comprises a plurality of drug microparticles selected from drug microspheres and drug microcapsules.

33. The tablet according to any one of claims 28-32, wherein the swellable component comprises a gellan gum.

34. The tablet according to any one of claims 28-32, wherein the swellable component comprises a gellan gum-high acyl.

35. The tablet according to any one of claims 28-34, wherein the tablet further comprises one or more hydrophilic agents.

36. The tablet according to claim 35, wherein the hydrophilic agent is an osmogen.

37. The tablet according to claim 36, wherein the osmogen is selected from the group consisting of magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, inulin, instant sugar, citric acid, succinic acid, tartaric acid, and mixtures thereof.

38. The tablet according to claim 36, wherein the osmogen is selected from lactose and mannitol.

39. The tablet according to claim 38, wherein the osmogen is the lactose product Capsulac 60.

40. The tablet according to any one of claims 28-39, wherein the tablet further comprises a tablet compression aid.

41. The tablet according to claim 40, wherein the tablet compression aid is silicified microcrystalline cellulose (SMCC).

42. The tablet according to any one of claims 28-41, wherein the tablet further comprises a hydrophilic agent selected from the group consisting of electrolytes, organic acids and mixtures thereof.

43. The tablet according to claim 42, wherein the electrolyte is calcium citrate.

44. The tablet according to claim 42, wherein the organic acid is citric acid.

45. The tablet according to claim 28, wherein the swellable component comprises gellan gum, a hydrophilic agent selected from lactose and mannitol and a compression aid.

46. The tablet according to claim 28, wherein the swellable component comprises gellan gum, a hydrophilic agent selected from lactose and mannitol and silicified microcrystalline cellulose.

47. The tablet according to claim 28, wherein the swellable component comprises gellan gum, a hydrophilic agent which is lactose and silicified microcrystalline cellulose.

48. The tablet according to any one of claims 45-47, wherein the drug-containing component comprises drug microspheres or drug microcapsules.

49. The tablet according to any one of claims 45-48, wherein the product is fully converted to a semi-solid form within about 90 seconds following the addition of water.

50. The tablet according to any one of claims 45-48, wherein the product is fully converted to a semi-solid form within about 45 seconds following the addition of water.

51. The solid pharmaceutical product according to claim 1 comprising a drug-containing component and a swellable component, wherein the product comprises gellan gum, a hydrophilic agent selected from lactose and mannitol and silicified microcrystalline cellulose.

52. The solid pharmaceutical product according to claim 1, wherein the drug-containing component comprises a plurality of drug mini-tablets and the swellable component comprises a plurality of swellable mini-tablets in which the swellable mini-tablets comprise gellan gum, a hydrophilic agent selected from lactose and mannitol and a compression aid.

53. The solid pharmaceutical product according to claim 1, wherein the drug-containing component comprises a plurality of drug mini-tablets and the swellable component comprises a plurality of swellable mini-tablets in which the swellable mini-tablets comprise gellan gum, a hydrophilic agent selected from lactose and mannitol and silicified microcrystalline cellulose.

54. The product according to any one of claims 51-53, wherein the product is fully converted to a semi-solid form within about 30 seconds following the addition of water.
